(19) United States
(12) Patent Application Publication
(10) Pub. No.: US 2004/0213854 A1
(43) Pub. Date: Oct. 28, 2004

(54) Granules otained by drying a mixture comprising a polymer and a control agent

(76) Inventors: Laurent Taisne, Paris (FR); Jean-Pierre Hecaen, Stains (FR)

Correspondence Address: Jean Louis Seugnet, Intellectual Property Department, Rhodia Inc CN 7500, 259 Prospect Plains Road, Cranbury, NJ, 08512-7500, US

(21) Appl. No.: 10/483,210

(22) PCT Filed: Jul. 12, 2002
(86) PCT No.: PCT/FR02/02480

(30) Foreign Application Priority Data
Jul. 12, 2001 (FR) 01 09391

(51) Int. Cl. A61K 9/14

(52) U.S. Cl. 424/489

(57) ABSTRACT

The invention concerns granules obtainable by drying an emulsion comprising at least an active substance; optionally a surfactant selected among non-ionic polyalkoxylated surfactants, or among anionic surfactants; at least a water-soluble or water-dispersible compound, selected among: (i) polymers obtained by polymerisation of at least an ethylenically unsaturated acid monomer and at least an ethylenically unsaturated hydrocarbon monomer; (ii) natural or synthetic polypeptides, optionally comprising at least a hydrophobic graft; (iii) highly depolymerized polysaccharides optionally comprising at least a hydrocarbon hydrophobic graft; at least a control agent selected among: complexing agents including at least one element of columns IIA, IVA, VA, VIII, IB, IIIB; cationic polymers. The inventive granules provide the advantage of enabling, once they are applied, gradual controlled or elicited release.

The present invention relates to granules that can be obtained by drying a mixture, in particular an emulsion comprising, inter alia, a control agent.

It is desirable in many fields to have solid formulations available comprising large amounts of active materials that can liberate them in a controlled manner, whether in the form of a progressive release over time and/or in the form of a release triggered by the appearance of a precise phenomenon. However, certain difficulties are encountered when the active materials that are to be incorporated are in the form of a hydrophobic liquid or in the form of a paste at ambient temperature. Methods exist that can be used to produce such solid formulations, for example by drying emulsions comprising, in addition to the active material, a surfactant and a compound that can obtain a solid matrix after eliminating the aqueous phase from the emulsion. While it is clear that such methods have constituted a technical advance, formulations, in particular concentrated formulations, are still not available that can progressively release over time and/or trigger the release of said active material.

The aim of the present invention is to provide granules that, with the same composition, can make available high concentrations of active material(s) in solid formulations, and also make possible a release that is spread over time and/or triggered from said active ingredient, once the formulation has been applied.

Thus, the present invention provides granules that can be obtained by drying a mixture comprising:

at least one active ingredient;

optionally, at least one surfactant selected from non ionic polyalkoxylated surfactants, anionic surfactants or mixtures thereof;

at least one hydrosoluble or hydrodispersible compound selected from:

(i) polymers obtained by polymerizing:

at least one acid monomer selected from linear or branched, aliphatic, cyclic or aromatic monocarboxylic or polycarboxylic acids carrying at least one ethylenically unsaturated bond; said monomers being used in the acid form or in the salt form, or in the form of macromonomers; and

at least one hydrocarbon monomer carrying at least one ethylenically unsaturated bond, in the form of a monomer or a macromonomer;

(ii) polypeptides of natural or synthetic origin, optionally comprising at least one hydrophobic graft containing 4 to 30 carbon atoms, which may be saturated or unsaturated, aromatic or non aromatic, optionally interrupted by one or more heteroatoms, and comprising at least one carboxylic acid and/or hydroxyl function;

(iii) highly depolymerized polysaccharides, optionally comprising at least one hydrophobic graft, containing 4 to 30 carbon atoms, which may be saturated or unsaturated, aromatic or non aromatic, optionally interrupted by one or more heteroatoms, and comprising at least one carboxylic acid and/or hydroxyl function.

In accordance with one characteristic of the invention, the emulsion comprises at least one control agent selected from:

complexing agents containing at least one element from columns IIA, IVA, VA, VIII, IB or IIIB;

cationic polymers.

The present invention also concerns the use of said granules as additives in formulations for use in the fields of foodstuffs, cosmetics, pharmaceuticals, detergents, paints, paper, agrochemicals, or metal working or deformation.

The granules of the invention have the advantage, when used, of reducing or eliminating the risks of transient overdosing of the active substances released into the medium. Thus, problems with toxicity or ecotoxicity are considerably lessened.

The granules of the invention can also increase the efficacy of a formulation because of the controlled release, progressive or triggered, of the active ingredient. As a result, the concentration of active ingredient in the medium to be treated is no longer zero or can be adapted to requirements.

Finally, the granules of the invention or formulations comprising them should be easier to use, since the user can space out the applications.

However, other characteristics and advantages of the present invention will become more apparent from the following description and examples.

The granules of the invention are obtained from a mixture that may be in the form of an aqueous solution or a direct oil-in-water emulsion, depending on whether the constituent elements are miscible or non miscible with the aqueous phase.

As indicated above, the granules comprise at least one active ingredient.

More particularly, this is selected from active ingredients that are in the form of a hydrophobic liquid or from non ionic surfactants and/or anionic surfactants, or mixtures thereof.

In a first implementation of the invention, the active ingredients employed are in the form of a liquid that is non miscible or very slightly miscible in water at ambient temperature, or in the dissolved form in an organic solvent, the ensemble being non miscible or very slightly miscible in water.

The term “slightly miscible” means active ingredients the solubility of which in water does not exceed 10% by weight between 20° C. and 30° C.

It should be noted that the present invention is also applicable to forming active ingredients with a melting point of 100° C. or less, more particularly 80° C. or less, and in particular in the range 20° C. to 80° C. As a result, the term “hydrophobic liquid” will also concern said active ingredients.

As a result, the term “active ingredient” as used in the present text means either the pure active ingredient, or the active ingredient dissolved in a solvent.

Active ingredients that can be employed in the granules of the invention can come from a wide variety of fields, such as foodstuffs, detergents, cosmetics, the pharmaceuticals industry, paints, paper, agrochemicals, working or deforming metals, etc.

Examples of active ingredients in the foodstuffs industry that can be cited are mono-, di- and tri-glycerides, essential oils, flavours, colorants and lipophilic vitamins.

Examples of active ingredients that can be used in the cosmetics field that can be cited are silicone oils, in particular those belonging to the dimethicone family; lipophilic vitamins such as vitamin A and its derivatives, vitamin B2, pantothenic acid, vitamin D and vitamin E; mono-, di- and tri-glycerides; fragrances; bactericides; UV absorbers such as PABA or PARA aminobenzoate type derivatives, salicylates, cinnamates, anthranilates, dibenzoylmethanes, camphor derivatives and mixtures thereof.

Anti-ageing agents can also be used. Examples of such agents that can be cited are retinoids, α- and β-hydroxy acids, their salts and their esters, liposoluble vitamins, ascorbyl palmitate, ceramides, pseudo-ceramides, phospholipids, fatty acids, fatty alcohols, cholesterol, sterols and mixtures thereof. Preferred fatty acids and alcohols that can in particular be cited are those with linear or branched alkyl chains containing 12 to 20 carbon atoms. It can in particular be linoleic acid.

It is also possible to employ anti-cellulite agents, such as isobutylmethylxanthine and theophylin; or anti-acne agents, such as resorcinol, resorcinol acetate, benzoyl peroxide and many natural compounds.

Flavours, fragrances, essential oils or essences, can also be used as the hydrophobic active ingredient. Examples that can be cited are oils and/or essences of mint, spearmint, peppermint, menthol, vanilla, cinnamon, bay, aniseed, eucalyptus, thyme, sage, cedar leaf, nutmeg, citrus (lemon, lime, grapefruit, orange), fruits (apple, pear, peach, cherry, plum, strawberry, raspberry, apricot, pineapple, grape, etc), used alone or as a mixture.

Anti-microbial agents can be selected from thymol, menthol, triclosan, 4-hexylresorcinol, phenol, eucalyptus oil, benzoic acid, benzoic peroxide, butyl paraben and mixtures thereof.

Examples of active ingredients that are suitable for the invention and can be used in the paints field that can be cited are alkyd resins, epoxy resins, and isocyanates, block or otherwise.

In the paper field, examples that can be cited include bonding and water-repelling resins such as alkylketene dimer (AKD) or alkenyl succinic anhydride (ASA).

In the agrochemicals field, a variety of plant health active ingredients can be employed.

Examples of active ingredients that are soluble or dissolved in the organic phase and which can be used in plant protective formulations that can be cited are active ingredients selected from the family of α-cyanophenoxybenzyl carboxylates or α-cyano-halogenophenoxycarboxylates, the family of N-methylcarbonates containing aromatic substituents, active ingredients such as Azinphos-methyl, Benfluralin, Bifenthrin, Chlorphoxim, Chlorpyrifos, Fluchloralin, Fluroxypyr, Dichlorvos, Malathion, Molinate, Parathion, Permethrin, Profenofos, Propiconazole, Prothiofos, Pyrifenox, Butachlor, Metolachlor, Chlorimephos, Diazinon, Fluazifop-P-butyl, Heptopargil, Macarbam, Proparfite, Prosulfocarb, Bromophosethyl, Carbophenothion, Cyhalothrin, Imazameth, Imazamthabenz, Imazamox, Imazapic, Imazapyr, Imazaquin, Imazethapyr, or mixtures thereof.

Suitable active ingredients for the field of plant protective formulations that can be cited are vegetable oils, mineral oils, silicone oils, silicone anti-foaming agents, etc.

In the detergents field, possible active ingredients that can be cited are aminated silicones as a softening agent, silicone anti-foaming agents, anti-microbial agents, fragrances, oils, essences, etc. In this respect, reference can be made to a list of compounds of this type indicated in the context of active ingredients for the cosmetics field.

Further suitable hydrophobic active ingredients that can be mentioned are:

organic oils/fats/waxes of animal origin or vegetable origin;

mineral oils/waxes;

products derived from the alcoholysis of said oils;

mono-, di- and tri-glycerides;

fatty acids, which may or may not be saturated, containing 10 to 40 carbon atoms;

esters of said acids and an alcohol containing 1 to 6 carbon atoms;

monoalcohols, which may or may not be saturated, containing 8 to 40 carbon atoms;

said compounds being used alone or as a mixture.

Organic oils/fats/waxes of animal origin that can be cited include sperm whale oil, whale oil, seal oil, shark oil, cod liver oil, pork fat, sheep fat (tallow), perhydrosqualene and beeswax, used alone or as a mixture.

Examples of organic oils/fats/waxes of vegetable origin that can be mentioned include rapeseed oil, sunflower seed oil, peanut oil, olive oil, hazelnut oil, corn oil, soya oil, avocado oil, linseed oil, hemp oil, grapeseed oil, coprah oil, African oil, cottonseed oil, palm nut oil, babassu oil, jojoba oil, sesame oil, castor oil, macadamia nut oil, sweet almond oil, camauba wax, shea butter, cocoa butter, peanut butter, used alone or as a mixture.

Mineral oils/waxes that can be cited include naphthene oils, paraffin oils (Vaseline), isoparaffins, paraffin waxes, used alone or as a mixture.

Products derived from the alcoholysis of said oils can also be used.

Regarding fatty acids, these latter, which may or may not be saturated, contain 10 to 40 carbon atoms, more particularly 18 to 40 carbon atoms, and can comprise one or more ethylenically unsaturated bonds, which may or may not be conjugated. It should be noted that said acids can comprise one or more hydroxyl groups.

Examples of saturated fatty acids that can be cited are palmitic acid, stearic acid, isostearic acid, behenic acid.

Examples of unsaturated fatty acids that can be cited are myristoleic, palmitoleic, oleic, erucic, linoleic, arachidonic, ricinoleic acid and mixtures thereof.

Fatty acid esters that can be cited include esters of the acids listed above, in which the portion deriving from the alcohol contains 1 to 6 carbon atoms, such as methyl, ethyl, propyl, isopropyl esters, etc.

In a second implementation of the invention, the active ingredient is selected from non ionic or anionic surfactants, or mixtures thereof.

More particularly, the non ionic surfactants are selected from non ionic polyalkoxylated surfactants such as:

polyalkoxylated fatty alcohols;

polyalkoxylated triglycerides;

polyalkoxylated fatty acid esters;

polyalkoxylated sorbitan esters;

polyalkoxylated fatty acid amides;

polyalkoxylated fatty amines;

polyalkoxylated amidoamines;

polyalkoxylated di(1-phenylethyl)phenols;

polyalkoxylated tri(1-phenylethyl)phenols;

polyalkoxylated alkylphenols;

polyalkoxylated polysiloxanes;

products resulting from the condensation of ethylene oxide or propylene oxide with ethylenediamine;

polyalkoxylated terpene hydrocarbons;

polyalkoxylated alkylpolyglycosides;

used alone or as a mixture.

The term (poly)alkoxylated means ethoxylated or propoxylated, motifs, or combinations thereof. Preferably, the surfactants include ethoxylated motifs, or ethoxylated/propoxylated motifs.

The number of ethoxylated motifs (OE) and/or propoxylated motifs (OP) in said surfactants is usually from 1 to 100, more particularly 1 to 50.

OE or OE/OP fatty alcohols generally contain 6 to 22 carbon atoms, the OE and OP motifs being excluded from these numbers. Preferably, said motifs are ethoxylated motifs (OE).

OE or OE/OP fatty acids particularly contain 6 to 22 carbon atoms, the OE and OP motifs being excluded from these numbers. Preferably, said motifs are ethoxylated motifs (OE).

More particularly OE or OE/OP triglycerides, preferably OE, are triglycerides of vegetable or animal origin, such as linseed oil, soya oil, castor oil, rapeseed oil, etc.

The acid portion of OE or OE/OP fatty acid esters generally contains 6 to 22 carbon atoms, the OE and OP motifs being excluded from these numbers, and are preferably ethoxylated (OE).

More particularly, OE or OE/OP sorbitan esters, preferably OE, are fatty acid esters of cyclized sorbitol containing 10 to 20 carbon atoms, such as lauric acid, stearic acid or oleic acid.

The term “OE or OE/OP triglyceride” as used in the present invention means both the products obtained by alkoxylation of a triglyceride by ethylene oxide and/or by propylene oxide as well as those obtained by transesterification of a triglyceride by a polyethylene glycol and/or polypropylene glycol.

Similarly, the term “OE or OE/OP fatty acid ester” includes products obtained by alkoxylation of a fatty acid by ethylene oxide and/or propylene oxide, as well as those obtained by transesterification of a fatty acid by a polyethylene glycol and/or polypropylene glycol.

OE or OE/OP amines and fatty amides generally contain 6 to 22 carbon atoms. OE and OP motifs being excluded from these numbers, and are preferably ethoxylated (OE).

OE or OE/OP amidoamines normally contain 2 to 22 carbon atoms as regards the hydrocarbon motifs, OE and OP motifs being excluded from these numbers, and are preferably ethoxylated (OE).

OE or OE/OP alkylphenols are conventionally 1 or 2 linear or branched alkyl groups containing 4 to 12 carbon atoms. Examples that can in particular be cited are octyl, nonyl or dodecyl groups.

More particularly, polysiloxanes, preferably ethoxylated (OE), are linear or branched polyalkylsiloxanes containing 2 to 10 silicon atoms, and in which the alkyl groups are preferably methyl radicals.

Suitable terpene hydrocarbons, preferably OE or OE/OP, are those derived from α- or β-pinenes. They have been described in International application WO-A-96/01246.

Alkylpolyglycosides can be obtained by condensing glucose with primary fatty alcohols containing a C₄-C₂₀ alkyl group as well as a mean number of glucose motifs of the order of 0.5 to 3 per mole of alkylpolyglycoside.

Regarding anionic surfactants, the following can in particular be cited:

alkylester sulphonates, for example with formula R—CH(SO₃M)—COOR′, where R represents a C₈-C₂₀ alkyl radical, preferably C₁₀-C₁₆, optionally carrying one or more unsaturated bonds, R′ represents a C₁-C₆ alkyl radical, preferably C₁-C₃, and M represents a hydrogen atom, an alkali cation (sodium, potassium, lithium) an alkaline-earth cation (for example calcium), substituted or unsubstituted ammonium (methyl-, dimethyl-, trimethyl-, tetramethylammonium, dimethylpiperidnium . . . ) or an alkanolamine derivative (monoethanolamine, diethanolamine, triethanolamine . . . ). More particularly, methyl ester sulphonates where radical R is C₁₄-C₁₆ can be cited;

alkylester sulphates, for example with formula R—CH(OSO₃M)-CH₂COOR′, in which R represents a C₈-C₂₀ hydrocarbon radical, preferably C₁₀-C₁₅, optionally carrying one or more saturated bonds, R′ is a C₁-C₆ alkyl radical, preferably C₁-C₃, and M has the meaning given above;

alkylbenzenesulphonates, more particularly C₉-C₂₀, primary or secondary alkylsulphonates, in particular C₈-C₂₂, alkylglycerol sulphonates;

alkylsulphates, for example with formula ROSO₃M, where R represents a C₁₀-C₂₄ alkyl or hydroxyalkyl radical, preferably C₁₂-C₂₀; M has the meaning given above;

alkylethersulphates, for example with formula RO(AO)ₙSO₃M, where R represents a C₁₀-C₂₄ alkyl or hydroxyalkyl radical, preferably C₁₂-C₂₀; OA represents an ethoxylated and/or propoxylated group; M has the meaning given above, n generally being between 1 and 4, such as laurylethersulphate where n=2;

alkylamide sulphates with formula RCONHR′OSO₃M, where R represents a C₂-C₂₂ alkyl radical, preferably C₆-C₂₀, R′ represents a C₂-C₃ alkyl radical, M has the meaning given above, and their polyaloxylated (ethoxylated and/or propoxylated) derivatives;

salts of saturated or unsaturated fatty acids, such as C₈-C₂₄ fatty acids, preferably C₁₄-C₂₀, and a cation having the same definition as M, N-acyl-N-alkyltaurates, alkylisethionates, alkylsuccinamates, monoesters or diesters of sulphosuccinates, N-acyl sarcosinates, polyethoxycarboxylates; and alkyl- or dialkylsulphosuccinates, for example C₆-C₂₁; the cation having the same definition as M; especially sodium dioctylsulposuccinate;

phosphate mono- and di-esters, for example with the following formula: (RO)_{x′}—P(═O))OM)ₓ, where R represents an alkyl, alkylaryl, arylalkyl, aryl radical, optionally polyalkoxylated, x and x′ being equal to 1 or 2, provided that the sum of x and x′ is equal to 3, M having the meaning given above; in particular, derivatives of polyalkoxylated fatty alcohols, polyalkoxylated di- and tri-(1-phenylethyl) phenols; polyalkoxylated alkylphenols; used alone or as a mixture.

As mentioned above, the active ingredients can be employed in the presence of a solvent for said active ingredients. More particularly, said solvent is selected from products that are not soluble or slightly miscible in water, as indicated above.

It should be noted that all of the hydrophobic liquid active ingredients that have been described can be used alone or as a mixture as a solvent for one or more other active ingredients, or one or more other surfactants.

It is also possible to use, as a solvent, aromatic oil cuts, terpene compounds such as D-limonene or L-limonene, and solvents such as Solvesso®. It is also possible to use aliphatic esters, such as methyl esters of a mixture of acetic acid, succinic acid and glutaric acid (mixture of acids as a by-product of Nylon synthesis), and chlorinated solvents.

It should be noted that the scope of the present invention includes combining various active ingredients, various surfactants, or associations thereof, provided that they are compatible.

The total quantity of active ingredient in the granules represents 30% to 90% dry weight. The emulsion from which the granules are obtained can also comprise at least one surfactant.

The surfactants cited above with regard to the possible active ingredients can be used as a surfactant.

When the active ingredient in the composition of the emulsion is selected from hydrophobic active ingredients, in other words from different species of surfactants, the surfactant, if present, is preferably selected from non ionic polyalkoxylated surfactants. Particularly advantageously, the surfactant is selected from polyalkoxylated sorbitan esters, polyalkoxylated triglycerides, used alone or as a mixture.

When the active ingredient is selected from hydrophobic active ingredients, the total amount of surfactant if present in the granules is in the range 0 (excluded) to 10% dry weight.

In accordance with the invention, one of the essential constituent elements of the emulsion from which the granules can be obtained is the hydrosoluble or hydrodispersible compound (hereinafter termed compound).

The term “hydrosoluble or hydrodispersible” means compounds which do not precipitate out or sediment out when in solution/dispersion in an aqueous phase, optionally with the surfactant, under the mixture preparation conditions, and in particular of the emulsion (concentration); the temperature being 20° C. to 30° C. The term “conditions for preparing the mixture and in particular the emulsion” means all or part of the range of concentrations of the compound in the final granule is in the range 10% to 70% of the dry granule weight. Further, this observation is made after the mixture (or emulsion) is left to rest (without stirring) for 10 minutes, between 20° C. and 30° C.

In accordance with a first implementation of the invention, the compound is selected from polymers obtained by polymerizing (i) at least one acid monomer selected from linear or branched, aliphatic, cyclic or aromatic monocarboxylic or polycarboxylic acids carrying at least one ethylenically unsaturated bond; said monomers being used in the acid form or in the salt form or in the form of a macromonomer; and at least one hydrocarbon monomer carrying at least one ethylenically unsaturated bond, in the monomeric form or as a macromonomer.

It should be remembered that the term “macromonomer” designates a macromolecule carrying one or more polymerizable functions.

More particularly regarding the acid monomer, it corresponds to the following formula:

(R′)HC═C(R²)COOM

in which formula:

R¹ represents a hydrogen atom, a —COOM group or a —CH₂)ₙ—COOM group in which n is in the range 1 to 4, a C₁-C₄ alkyl radical; R² represents a hydrogen atom, a —CH₂)ₘ—COOM group in which m is in the range 1 to 4, or a C₁-C₄ alkyl group; M represents a hydrogen atom, an alkali metal (such as sodium, potassium, lithium, magnesium) or a NR₄⁺ type ammonium group in which R, which may be identical or different, represents a hydrogen atom, an alkyl radical containing 1 to 4 carbon atoms, preferably 1 or 2 carbon atoms, which may or may not be substituted by an oxygen atom; said monomer being used alone or as a mixture, or in the form of macromonomers of one or more thereof.

Preferably, said acid monomer is such that the radical R¹ represents a hydrogen atom, a —COOM group or —(CH₂)—COOM group, a methyl radical, and radical R represents a hydrogen atom, a —CH₂—COOM group or a methyl radical.

In accordance with a highly advantageous implementation of the present invention, the acid monomer is selected from acrylic, methacrylic, citraconic, maleic, fumaric, itaconic, or crotonic acid or anhydride, used alone or as a mixture, their salts, or in the form of macromonomers of one or more thereof.

Regarding the hydrocarbon monomer, this latter more particularly corresponds to the following formula:

(R²)(R²)—C═CH₂ (II)

in which formula radicals R², which may be identical or different, represent a hydrogen atom, a linear or branched, cyclic or cycloaliphatic C₁-C₁₀ aliphatic radical, optionally carrying an ethylenically unsaturated bond; or an aromatic radical, preferably containing 6 carbon atoms, optionally substituted with at least one C₁-C₁₀ alkyl radical, optionally carrying an ethylenicallv unsaturated bond. Again, said monomers can be used alone or as a mixture, or in the form ot macromonomers of one or more thereof.

Said hydrocarbon monomer can advantageously be selected from ethylene, propylene. 1-butene, isobutylene, n−1-pentene, 2-methyl-1-butene, n−1-hexene, 2-methyl-1-pentene, 4-methyl-1-pentene, 2-ethyl-1-butene, diisobutylene, 2-methyl-3,3-dimethyl-1-pentene, styrene, α-methylstyrene, vinyltoluene, butadiene, chloroprene, isoprene, or mixtures thereof, and macromonomers deriving from said monomers.

Preferably, radicals R², which may be identical or different, represent a hydrogen atom, or a saturated, linear or branched aliphatic, cyclic or cycloaliphatic C₁-C₁₀ radical.

The monomers and their respective proportions are selected so that the resulting copolymer is hydrosoluble or hydrodispersible.

Preferably, the distribution of the monomers in the copolymer is random. Clearly, the scope of the present invention encompasses using block copolymers.

The compounds employed in the emulsion are copolymers that are well known to the skilled person and are prepared prior to introducing them into the mixture, in particular the emulsion. They are conventionally obtained by using radical polymerization, conventional or controlled, or anionic in type.

Copolymers of this type that can be cited are those obtained from maleic anhydride and an olefin.

In a second variation of the invention, the compound is selected from polypeptides of natural or synthetic origin, comprising at least one carboxylic acid function and/or at least one hydroxyl function.

More particularly, the polypeptides are selected from:

polypeptides of vegetable origin such as proteins deriving from proteaginous grain (in particular peas, beans, lupin, haricot and lentil); proteins deriving from cereal grain (such as those from wheat, barley, rye, corn, rice, oats, millet); proteins deriving from oleaginous grain (such as soya, peanut, sunflower, rapeseed and coconut); proteins deriving from leaves (alfalfa and nettles): proteins deriving from plant parts and buried reserves (for example potato, beet); and their hydrolysates;

polypeptides of animal origin such as muscle protein; proteins deriving from milk (such as casein, lactoglobulin); fish protein; and their hydrolysates.

More particularly, said polypeptides are hydrosoluble or hydrodispersible polymers, as defined above.

Further, the degree of hydrolysis of said proteins is 40% or less.

Said polymers are homopolymers or copolymers derived from the polycondensation of aminated acids, in particular aspartic acid, and/or glutamic acid or other copolymerizable amino acids, such as glycine, alanine, leucine, isoleucine, phenylalanine, methionine, histidine, proline, lysine, serine, threonine, cysteine, etc.

In a third variation of the present invention, the compound is selected from highly depolymerized polysaccharides, comprising at least one carboxylic acid function and/or at least one hydroxyl function.

More precisely, said highly depolymerized polysaccharides are obtained from:

polysaccharides of bacterial origin, such as xanthan gum, succinoglycans;

polysaccharides of animal or vegetable origin such as carrageens, galactomannans (for example guar, carob), glucomannans, cellulose, maltodextrins; alone or as a mixture.

Polymers are termed “highly depolymerized” within the context of the present invention, when their mass average molar mass is 50000 g/mol or less, preferably 20000 g/mol (absolute value determined by MALLS (multiple angle laser light scattering) coupled with gel permeation chromatography, or by nuclear magnetic resonance).

Non limiting examples of highly depolymerized polysaccharides that can be cited are those obtained from starch, maltodextrins, xanthan gum and galactomannans such as guar or carob.

The compounds of the second and third variations can optionally comprise hydrophobic grafts. Said grafts can be bound to the polypeptide or to the polysaccharide via amide, ester urea, urethane, isocyanate, amino bonds. Said graft polymers are, for example, obtained bN reacting a portion of the amino acid functions or free alcohols with compounds that can create said bonds.

Regarding the hydrophobic graft, if present, it is more particularly selected from aliphatic, cyclic, aromatic, alkylaromatic and arylaliphatic radicals containing 4 to 30 carbon atoms, and can be interrupted by one or more heteroatoms such as oxygen or nitrogen.

Here again, the molar masses of the polymers and the respective proportions of the monomers present and optional hydrophobic grafts are such that the polymer concerned is hydrosoluble or hydrodispersible.

By way of indication, the mass average molar mass of the polymers in the above three variations is more particularly 50000 g/mol or less, advantageously 20000 g/mole or less (absolute value determined by MALLS (multiangle laser light scattering) coupled with gel permeation chromatography, or by NMR).

Said hydrosoluble and/or hydrodispersible compounds have been described in International patent application WO-A-00/26280.

In an advantageous implementation of the present invention, the amount of compound in the granule represents 10% to 70% of the dry weight.

Advantageously, when a granule comprises at least one hydrophobic liquid active ingredient selected from different species of surfactants and a surfactant, the amount of compound is such that the weight ratio of the concentrations [surfactant(s)/compound] is in the range 1/99 to 70/30, more particularly in the range 1/99 to 50/50, preferably 1/99 to 30/70.

In accordance with one characteristic of the invention, the mixture (in particular the emulsion) from which the granules are obtained further comprises at least one control agent selected from:

complexing agents comprising at least one element from columns IIA, IVA, VA, VIII, IB and IIIB of the periodic table published in the Bulletin de la Société Chimique de France, n^{o} 1, January 1966);

cationic polymers.

More particularly, the complexing agents are in the ionic, anionic or cationic form, or in the form of oxide or hydroxide particles. In this latter case, the size of the oxide particles is nanometric (more particularly less than 200 nm, preferably 100 nm or less).

In accordance with a particular implementation of the invention, the control agent selected from complexing agents comprises at least one element selected from calcium, magnesium, titanium, zirconium, copper, vanadium, iron, cobalt, aluminium, boron, and mixtures thereof.

The counter-ions for the complexing agents described above are preferably selected from mineral or organic ions which produce species that are soluble in aqueous media, when combined with the complexing agent.

Examples of anionic mineral counter-ions that can be cited are halides, such as chlorides, carbonates, nitrates, sulphates, hydrosulphates, alkylsulphates (for example containing 1 to 6 carbon atoms), phosphates, citrates, formates and acetates.

Examples of anionic organic counter-ions that can be mentioned are acetates, formates and lactates.

Examples of cationic counter-ions that can be cited are alkali metals, NR₄⁺ type ammonium ions in which R, which may be identical or different, represent a hydrogen atom or an alkyl radical containing 1 to 4 carbon atoms, preferably 1 or 2 carbon atoms, which may or may not be substituted with an oxygen atom.

Depending on the nature of the elements, the complexing agents can create ionic interactions with the compound. This is the case, for example, with ions of the borate, aluminate, vanadate, vanadyl sulphate, titanate, titanyl sulphate type, etc.

The complexing agents can also create interactions of the hydrogen bond type with the compound in the oxide or hydroxide form, for example.

More particularly, the mole ratio [number of metal atoms/number of hydroxyl functions and/or carboxylic functions of the compound] is in the range 1/1 to 1/100.

In accordance with a second possibility, the control agent is selected from cationic polymers.

Advantageously, these latter are selected from polyvinylpyrrolidone, cationic guars, cationic celluloses, cationic starches and synthetic polymers the net charge of which is cationic.

It should be remembered that the net charge represents the difference in the positive and negative charges of the polymer, under the pH conditions of the emulsion.

Regarding guars, celluloses, cationic starches, the cationic group is more particularly a quaternary ammonium group carrying three radicals, which may or may not be identical, selected from hydrogen and an alkyl radical containing 1 to 22 carbon atoms, more particularly 1 to 14, advantageously 1 to 3 carbon atoms.

Regarding the counter-ion, this latter can be selected from halides such as chlorides, carbonates, nitrates, sulphates, hydrosulphates, alkylsulphates (for example containing 1 to 6 carbon atoms), phosphates, citrates, formates, acetates.

It should be noted that the scope of the present invention encompasses employing cationic polymers comprising hydrophobic groups such as C₁-C₁₄ alkyl chains, preferably C₂-C₈, optionally having a hydroxyl group. Said hydrophobic groups are connected to the principal polymeric chain, in particular via ether bonds.

Regarding synthetic cationic polymers, we can cite those obtained by polymerizing at least one monomer selected from:

aminoalkyl (meth)acrylates, aminoalkyl (meth)acrylamides;

monomers comprising at least one secondary, tertiary or quaternary amine function, or a heterocyclic group containing a nitrogen atom, vinylamine, ethylene imine;

diallyldialkyl ammonium salts;

alone or as a mixture, their salts, and macromonomers deriving from said monomers.

It should be stated that under the pH conditions of the mixture (and in particular the emulsion), the polymers have cationic charges.

Suitable monomers that can be cited are:

dimethylaminoethyl(meth)acrylate, dimethylaminopropyl(meth)acrylate, ditertiobutylaminoethyl(meth)acrylate, dimethylaminomethyl(meth)acrylamide, dimethylaminopropyl(meth)acrylamide;

ethylene imine, vinylamine, 2-vinylpyridine, 4-vinylpyridine;

trimethylammonium ethyl(meth)acrylate chloride, trimethylammonium ethyl acrylate methyl sulphate, benzyl dimethylammonium ethyl(meth)acrylate chloride, 4-benzoylbenzyldimethylammonium ethyl acrylate chloride, trimethylammonium ethyl(meth)acrylamidochloride, trimethylammonium vinylbenzyl chloride;

diallylmethyl ammonium chloride;

alone or as a mixture, their salts, as well as macromonomers deriving from said monomers.

The counter-ion for said monomers can be selected from halides such as chlorides, carbonates, nitrates, sulphates, hydrosulphates, alkylsulphates (for example containing 1 to 6 carbon atoms), phosphates, citrates, formates, acetates.

Said synthetic cationic polymers can comprise one or more anionic motifs under the mixture preparation conditions (in particular of the emulsion) provided that the net charge of the polymer is positive.

Advantageously, the anionic monomers can be selected from those carrying at least one carboxylic, sulphonic, sulphuric, phosphonic, phosphoric, sulphosuccinic function, and the corresponding salts.

More particularly, said monomers can be selected from:

linear or branched, cyclic or aromatic mono- or poly-carboxylic acids, N-substituted derivatives of said acids, polycarboxylic acid monoesters, comprising at least one ethylenically unsaturated bond;

linear or branched, cyclic or aromatic carboxylic vinyl acids;

amino acids containing at least one ethylenically unsaturated bond, amino acid N-carboxy anhydrides;

alone or as a mixture, their sulphonic or phosphonic derivatives, macromonomers of said monomers, or its corresponding salts.

Particular non limiting examples of monomers that can be used that can be cited are:

acrylic acid, methacrylic acid, fumaric acid, itaconic acid, citraconic acid, maleic acid, oleic acid, linoleic acid, linolenic acid, acrylamidoglycolic acid, 2-propene-1-sulphonic acid, methallylsulphonic acid, styrenesulphonic acid, α-acrylamidomethylpropanesulphonic acid, 2-sulphoethylene methacrylate, sulphopropylacrylic acid, bis-sulphopropylacrylic acid, bis-sulphopropylmethacrylic acid, sulphatoethyl methacrylic acid, the phosphate monoester of hydroxyethyl methacrylic acid, and their alkali metal salts, such as sodium or potassium salts, or ammonium salts;

vinyl sulphonic acid, vinylbenzene sulphonic acid, vinylphosphonic acid, vinylidene phosphoric acid, vinylbenzoic acid, and their alkali metal salts such as sodium or potassium salts, or ammonium salts;

N-methacryloyl alanine, N-acryloyl-hydroxy-glycine;

alone or as a mixture, as well as macromonomers deriving from said monomers, or the corresponding salts.

More particularly, the salts are alkali metal salts, or NR₄⁺ type ammonium salts in which R, which may be identical or different, represents a hydrogen atom or an alkyl radical containing 1 to 4 carbon atoms, preferably 1 to 2 carbon atoms, which may or may not be substituted by an oxygen atom.

The polymers can also comprise one or more monomers carrying no ionic charge under the mixture conditions (in particular of the emulsion). Depending on the pH range of the mixture (or emulsion), certain monomers listed in the context of cationic or anionic monomers may not have an ionic charge.

It is also possible to use monomers which, whatever the pH conditions of the emulsion, carry no ionic charge. Particular examples of monomers of this type that are suitable are:

ethylene oxide;

esters of (meth)acrylic acid with an alcohol containing 1 to 12 carbon atoms, such as methyl (meth)acrylate, ethyl (meth)acrylate, propyl (meth)acrylate, n-butyl (meth)acrylate, t-butyl (meth)acrylate, t-butyl (meth)acrylate, isobutyl (meth)acrylate, 2-ethylhexyl acrylate, hydroxyethyl (meth)acrylate;

vinyl acetate (to obtain polyvinyl alcohol which is partially or completely deacetylated), vinyl Versatate®, vinyl propionate, vinyl chloride, vinylidene chloride, methyl vinylether, ethyl vinylether; (meth)acrylonitrile, N-vinylpyrrolidone, vinylformamide, vinylacetamide;

(meth)acrylamide, N-alkyl (meth)acrylamide such as isopropyl acrylamide, N-methylol(meth)acrylamide;

styrene, α-methylstyrene, vinyltoluene, butadiene, chloroprene, isoprene;

alone or as a mixture, and macromonomers deriving from said monomers.

Preferably, the cationic polymers employed are copolymers containing at least one cationic monomer and at least one anionic monomer under the pH conditions of the mixture (in particular the emulsion), it being understood that the net charge of said polymer is a cationic charge.

The polymers used may or may not have a random monomer distribution.

Such polymers can in particular be obtained by radical polymerization, controlled or otherwise, in a manner conventional to the skilled person.

Regarding controlled radical synthesis, reference can in particular be made to the processes described in the following applications: WO-A-98/58974, WO-A-00/75207 and WO-A-01/42312 (xanthate type control agents), WO-A-98/01478 (dithioester type control agents), WO-A-99/03894 (nitroxide precursors), WO-A-99/31144 (dithiocarbamate type control agents), WO-A-02/26836 (dithiocarbazate type control agents), WO-A-02/10223 (dithiophosphoroester type control agents), WO-A-96/30421 (atom transfer radical polymerization—ATRP).

Preferably, the quantity of cationic polymer is such that the mole ratio [number of cationic charges/number of hydroxyl functions and/or carboxylic functions of the compound] is in the range 1/1 to 1/100.

The redispersible granules of the present invention can also contain at least one supplemental ionic surfactant.

More particularly, the supplemental ionic surfactants can be amphoteric surfactants, alkyl betaines, alkyldimethylbetaines, alkylamidopropylbetaines, alkylamidopropyldimethylbetaines, alkyltrimethylsulphobetaines, imidazoline derivatives such as alkylamphoacetates, alkylamphodiacetates, alkylamphopropionates, alkylamphodipropionates, alkylsultaines or alkylamidopropylhydroxysultaines, the condensation products of fatty acids and protein hydrolysates, amphoteric derivatives of alkylpolyamines such as Amphionic XL® sold by Rhodia, Ampholac 7T/X® and Ampholac 7C/X® sold by Berol Nobel.

The cation is generally an alkali or alkaline-earth metal such as sodium, potassium, lithium or magnesium, or an NR₄⁺ ammonium group in which R, which may be identical or different, represent a hydrogen atom, an alkyl radical containing 1 to 4 carbon atoms, preferably 1 to 2 carbon atoms, substituted or not substituted by oxygen or nitrogen.

It is possible to add to the redispersible granules of the invention any additive that is conventional in the field of application of the latter, such as anti-clumping agents, wetting agents, disintegrating agents, etc.

As indicated above, the granules of the invention are capable of being obtained by carrying out the following steps:

preparing a mixture from an aqueous solution comprising the compound, optional surfactant and active ingredient;

adding the control agent to the mixture obtained;

drying the resulting mixture.

When an emulsion is obtained during the first step, it is a direct oil-in-water emulsion.

According to a preferred implementation of the invention, when one or more liquid hydrophobic active ingredients selected from different species of surfactants are used in association with at least one surfactant, then a solution of the active ingredient(s) is prepared, optionally in a form that is dissolved in a suitable solvent. Further, an aqueous solution is prepared comprising the surfactant and the compound. Then, the aqueous solution is added to the solution of hydrophobic active ingredient(s), with stirring. In this case, a direct emulsion is obtained.

The mixture, possibly an emulsion, is more particularly prepared at a temperature of less than 100° C. and is preferably in the range 20° C. to 90° C. It should be noted that the temperature at which the mixture (or emulsion) is prepared is such that the various ingredients are in the liquid form. In the case in which a hydrophobic active ingredient is present, the temperature conditions result in the production of an emulsion.

When an emulsion is obtained, the mean droplet size (d₅₀) is in general in the range 0.1 to 10 micrometers and preferably between 0.2 and 5 micrometers (light diffusion observation).

The quantity of dry matter in the mixture (and in particular the emulsion) is generally in the range 10% to 80% by weight.

The respective amounts of the various constituents in the mixture (or emulsion) are selected so that the dry granules have the composition defined above.

Once the mixture (or emulsion) is obtained, a control agent is added which is preferably in the form of an aqueous solution.

The third step of the preparation process of the invention consists of drying the mixture (or emulsion) so formulated to obtain granules.

The method employed to eliminate water from the mixture (or emulsion) and obtain granules can be carried out by any means known to the skilled person.

This operation takes place so that the various constituent elements of the mixture are subjected to temperatures that are below the degradation temperature.

In a first implementation of the invention, it is possible to envisage oven drying. Preferably, drying takes place in a thin layer. More particularly, the temperature at which drying is carried out is 100° C. or less, preferably in the range 50° C. to 90° C.

In accordance with a further particular implementation of the invention, rapid drying is carried out on the mixture (or emulsion). Spray drying is suitable in this context, in a fluidized bed, using Duprat® drums or freeze-drying (freezing-sublimation).

Spray drying or fluidized bed drying can normally be carried out in any known apparatus such as a spray tower, which combines spraying carried out via a nozzle or a turbine with a stream of hot gas. The hot gas inlet temperature (generally air) at the column head is preferably in the range 100° C. to 250° C. and the outlet temperature is preferably below the degradation temperature of the constituent elements of the granules obtained.

In the case of operations for drying the mixture (or emulsion) carried out using the Duprat® drum or any means that can rapidly produce a dry film which is separated from the drying support by a raking operation, for example, to obtain particles which can optionilly be ground. If necessary, said particles can subsequently be formed, such as an agglomeration step, to obtain granules.

It should be noted that additives such as anticlumping agents can be incorporated into the granules at the time of this drying step.

It is recommended, for example, that a filler selected from calcium carbonate, barium sulphate, kaolin, silica, bentonite, titanium oxide, talc, hydrated alumina and calcium sulphoaluminate could be used.

The granules of the invention can be used in a wide variety of fields. They can be used as additives in formulations that can be used in the fields of foodstuffs, detergents, cosmetics, the pharmaceutical industry, paints, paper, agrochemicals and metal working or deformation.

A non limiting example of the invention will now be given.

### EXAMPLE

For 165 g of dry granules. the composition was as follows:

| Product | Quantity | |
|---|---|---|
| Geropon EGPM (sold by Rhodia Geronazzo) | 325.7 | g |
| Maleic anhydride/olefin copolymer (in | | |
| aqueous solution, about 26%) | | |
| Rapeseed oil | 40 | g |
| Alkamuls B (sold by Rhodia Chimie) | 5 | g |
| 33 OE castor oil | | |
| Aquarhône (sold by Rhodia Chimie) | 80.4 | g |
| Aluminium polychloride (in aqueous solution, 9% aluminium) | | |
| Sodium hydroxide (30%) | 37.5 | g |

NB: the quantities are expressed as the dry weight.

Obtaining Granules:

Firstly, the aqueous phase was obtained by dissolving the polymer in water, and sodium hydroxide was added.

The rapeseed oil, in which the surfactant had been dissolved, was then added.

The ensemble was stirred at 1000 rpm for 10 minutes.

The cross-linking agent (Aquarhône) was slowly added with stirring. The pH of the emulsion was about 7.

The emulsion obtained was oven dried in a thin film (12 hours, 75° C.).

The film was then ground to obtain a powder which was sieved (size below 400 μm).

Dissolution Test:

Three samples were prepared which were brought into contact with an aqueous solution at a variable pH and dissolution of the powders was observed.

| pH 10 | pH 10.5 | pH 11 |
|---|---|---|
| Insoluble | Partially soluble | Completely soluble |

1-18. Canceled

19. Granules made by the process comprising the steps of:
a) drying a mixture comprising:
at least one active ingredient;
optionally, at least one surfactant selected from non ionic polyalkoxylated surfactants or from anionic surfactants;
at least one hydrosoluble or hydrodispersible compound which is:
a polymer (i) obtained by polymerizing:
at least one acid monomer which is a linear or branched, aliphatic, cyclic or aromatic monocarboxylic or polycarboxylic acid carrying at least one ethylenically unsaturated bond; said monomer being used in the acid form or in the salt form, or in the form of macromonomer; and
at least one hydrocarbon monomer carrying at least one ethylenically unsaturated bond, in the form of a monomer or a macromonomer;
a polypeptide (ii) of natural or synthetic origin, optionally comprising at least one hydrophobic hydrocarbon graft, containing 4 to 30 carbon atoms, saturated or unsaturated, aromatic or non aromatic, optionally interrupted by one or more heteroatoms, and comprising at least one carboxylic acid or at least one hydroxyl function; or
a highly depolymerized polysaccharide (iii), optionally comprising at least one hydrophobic hydrocarbon graft containing 4 to 30 carbon atoms, saturated or unsaturated, aromatic or non aromatic, optionally interrupted by one or more heteroatoms, and comprising at least one carboxylic acid or hydroxyl function;
said emulsion furthercomprising at least one control agent selected from the group consisting of:
complexing agents containing at least one element from columns IIA, IVA, VA, VIII, IB or IIIB; and
cationic polymers.

20. Granules according to claim 19, wherein the active ingredient is in the form of a hydrophobic liquid, from non ionic surfactants or anionic surfactants.

21. Granules according to claim 19, wherein the mixture is an emulsion.

22. Granules according to claim 19, wherein the polyalkoxylated non ionic surfactant is a:
polyalkoxylated fatty alcohol;
polyalkoxylated triglyceride;
polyalkoxylated fatty acid ester;
polyalkoxylated sorbitan ester;
polyalkoxylated fatty acid amide;
polyalkoxylated fatty amine;
polyalkoxylated amidoamine;
polyalkoxylated di(1-phenylethyl)phenol;
polyalkoxylated tri(1-phenylethyl)phenol;
polyalkoxylated alkylphenol;
polyalkoxylated polysiloxane;
product resulting from the condensation of ethylene oxide or propylene oxide with ethylene diamine;
polyalkoxylated terpene hydrocarbon; or
polyalkoxylated alkylpolyglycoside.

23. Granules according to claim 19, wherein the anionic surfactant is a:
alkylester sulphonate;
alkylester sulphate;
alkylbenzenesulphonate, primary or secondary alkylsulphonate, alkylglycerol sulphonate;
alkylsulphate;
alkylethersulphate;
alkylamide sulphate;
salt of saturated or unsaturated fatty acids, N-acyl N-alkyltaurates, alkylisethionates, alkylsuccinamates, monoesters or diesters of sulphosuccinates, N-acyl sarcosinates, polyethoxycarboxylates;
alkyl or dialkyl-sulphosuccinate; or
phosphate mono- or di-ester.

24. Granules according to claim 19, wherein the active ingredient is present in the granule in a total quantity of 30% to 90% of the dry weight.

25. Granules according to claim 19, wherein the active ingredient is an hydrophobic active ingredient, with a total surfactant content in the granule in the range from 0 (excluded) to 10% of the dry weight.

26. Granules according to claim 19, wherein the polymer (i) is obtained by polymerizing at least one acid monomer corresponding to the following formula:
(R¹)HC═C(R²)COOM
wherein:
R¹ represents a hydrogen atom, a —COOM group or a —(CH₂)ₙ—COOM group in which n is in the range 1 to 4, or a C₁-C₄ alkyl radical; R² represents a hydrogen atom, a —(CH₂)ₘ—COOM group in which m is in the range 1 to 4, or a C₁-C₄ alkyl group; M represents a hydrogen atom, an alkali metal, an alkaline-earth metal or a NR₄⁺ type ammonium group in which R, which are identical or different, represents a hydrogen atom, an alkyl radical containing 1 to 4 carbon atoms, which are optionally substituted with an oxygen atom.

27. Granules according to claim 26, wherein the hydrocarbon monomer corresponds to the following formula:
(R²)(R²)—C═CH₂ (II)
wherein radicals R², which are identical or different, represent a hydrogen atom, a linear or branched, cyclic or cycloaliphatic C₁-C₁₀ aliphatic radical, optionally carrying an ethylenically unsaturated bond.

28. Granules according to claim 19, wherein the polypeptide (ii) is:
a protein deriving from proteaginous grain; cereal grain; oleaginous grain; leaves;
plant parts or buried reserves; or
a protein deriving from muscle, milk or fish.

29. Granules according to claim 19, wherein the highly depolymerized polysaccharide (iii) is obtained from:
xanthan gum, succinoglycan, carrageen, galactomannan, glucomannan, cellulose or maltodextrins.

30. Granules according to claim 19, wherein the control agent is a complexing agent comprising calcium, magnesium, titanium, zirconium, copper, vanadium, iron, cobalt, aluminium, or boron.

31. Granules according to claim 19, wherein the compound presents a mole ratio [number of metal atoms/number of hydroxyl functions and/or carboxylic functions of the compound] in the range 1/1 to 1/100.

32. Granules according to claim 19, wherein the control agent is a cationic polymer obtained by polymerizing at least one of the following monomers:
aminoalkyl (meth)acrylates, aminoalkyl (meth)acrylamides;
monomers comprising at least one secondary, tertiary or quaternary amine function, or a heterocyclic group containing a nitrogen atom, vinylamine, ethylene imine; or
diallyldialkyl ammonium salts.

33. Granules according to claim 19, wherein the mixture presents a mole ratio [number of cationic charges/number of hydroxyl functions and/or carboxylic functions of the compound] in the range 1/1 to 1/100.

34. Granules as defined in claim 19, made by the process comprising the following steps:
1) preparing a mixture from an aqueous solution comprising the compound, optional surfactant, and active ingredient;
2) adding the control agent to the mixture obtained in step 1); and
3) drying the resulting mixture obtained in step 2).

35. Granules according to claim 34, wherein the drying is carried out in an oven, in a thin film, by spray drying, or in a fluidized bed.

36. Additives in formulations for foodstuffs, detergents, cosmetics, the pharmaceutical industry, paints, paper, agrochemicals, metal working or deformation comprising granules as defined in claim 19.
